(19) United States
(12) Patent Application Publication
(10) Pub. No.: US 2007/0117828 A1
(43) Pub. Date: May 24, 2007

(54) Pharmaceutical compositions

(75) Inventors: Robert D. Simmons, Martinsville, NJ (US); Allan J. Weingarten, Westfield, NJ (US); Yuping Li, Bridgewater, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering-Plough Animal Health Corp.

(21) Appl. No.: 11/600,560

(22) Filed: Nov. 16, 2006

Related U.S. Application Data
(60) Provisional application No. 60/738,524, filed on Nov. 21, 2005.

(51) Int. Cl. A61K 31/485

(52) U.S. Cl. 514/282

(57) ABSTRACT

A method of providing systemic analgesia to cats, dogs and other small mammals by the otic or transdermal administration of opioids is disclosed. Compositions for use in such a method are also disclosed.

### CROSS REFERENCE TO RELATED APPLICATIONS

This application is a non-provisional application that claims the benefit of priority under 35 U.S.C. § 119(e) of provisional application U.S. Ser. No. 60/738,524 filed Nov. 21, 2005, the contents of which is hereby incorporated by reference in its entirety.

### FIELD OF THE INVENTION

The present invention relates to compositions and methods for providing systemic analgesia, and more particularly to the otic and transdermal administration of opioid analgesics to cats, dogs and other mammals.

### BACKGROUND OF THE INVENTION

All patents, applications, publications, test methods, and other materials cited herein are incorporated by reference.

Pain activates the stress hormone systems of the body and contributes to morbidity and mortality. Relief of pain (analgesia) in animals can safely be provided by opioids titrated to effect. Opioids can provide profound analgesia with minimal cardiovascular side effects, are safe alone and in combination with anesthetics, and are reversible if an adverse event should occur.

Historically, pharmacologic agents, including opioids, have been administered through systemic injection (subcutaneous, intramuscular or intravenous), epidurally, intrathecally (into the subarachnoid space), sublingually, orally, rectally and transdermally to provide analgesia. With the exception of epidural and intrathecal delivery, administration of these agents provides systemic drug delivery to produce analgesic effects. Epidural and intrathecal administration involves the direct administration of an analgesic agent to receptors in the spinal cord involved in spinal transmission of pain (e.g. opioid receptors), bypassing the need for systemic exposure to the pharmacologic agent in question.

Opioids produce analgesia by binding with opioid receptors within the nervous system to block the transmission of the pain impulse to the higher brain centers, thus diminishing or blocking the perception of pain. There are three types of well-characterized opioid receptors: mu, kappa and delta. Most of the clinically useful opioid medications are mu agonists.

TORBUGESIC-SA (butorphanol tartrate) is a veterinary product approved in the U.S. for perioperative analgesia. Butorphanol is an opioid agonist/antagonist.

Full opioid agonists such as oxymorphone, morphine, meperidine and fentanyl can provide profound analgesia to animals and are safe for use in combination with anaesthetics. For example, hydromorphone is used in veterinary practice as a perioperative analgesic by the injectable route of administration. However, parenteral administration is not practical for use by animal owners without veterinary training. Oral formulations of many opioids are also available, but opioid agonists have a low systemic bioavailability when administered orally due to extensive hepatic first-pass metabolism. Fentanyl has been administered transdermally via adhesive drug-filled patches, but such patches are expensive and inconvenient to use on fur-covered animals. Moreover, transdermal patches require up to six hours to achieve a therapeutic effect, and analgesia must be provided by other means in the interim.

In addition to the shortcomings of present methods for the administration of opioids to animals discussed above, the possibility of overdose and the potential for abuse by humans has limited their use in animals.

U.S. Pat. No. 5,589,480 relates to a method for inducing analgesia in inflamed skin by topically administering to the skin an opioid analgesic agent in an amount that is ineffective for induction of systemic analgesia. According to this patent, effective analgesia must be induced in the “substantial absence of transdermal delivery of the opioid analgesic agent to the systemic circulation.” U.S. Pat. No. 6,011,022 relates to a method of inducing analgesia in skin or mucosal tissue, comprising ocularly administering an analgesic agent that affects peripheral muscarinic receptors, which amount is systemically ineffective for induction of analgesia, and whereby the analgesia of the skin or mucosal tissue is induced in the substantial absence of transdermal or transmucosal delivery of the analgesic agent to the central nervous system. While oxymorphone and morphine are mentioned as analgesic agents that may be used in conjunction with a muscarinic receptor agonist analgesic, they are not themselves muscarinic receptor agonists. “Mucosal tissue” is specifically defined in the specification as excluding the conjunctiva of the eye.

The administration of certain veterinary drugs by the otic route is also known, but not for the provision of systemic analgesia. For example, methimazole is administered to the ear pinnae of cats to control hyperthyroidism. U.S. Pat. No. 5,543,434 relates to the nasal or ocular administration of ketamine to control chronic pain. U.S. Pat. No. 6,191,126 B1 is directed to the administration of kappa opioid agonists to the eye to treat ocular pain. This patent stresses that kappa opioids act on receptors in peripheral tissue, while mu opioids relieve pain by activating receptors in the brain. The local action of kappa opioids is said to be an advantage over systemic action. Accordingly, this invention is only suitable for treatment of pain in the ophthalmic tissues, not systemic analgesia.

In view of the foregoing limitations and shortcomings of the prior art formulations and methods, as well as other disadvantages not specifically mentioned above, it is apparent that there still exists a need in the art for improved means for systemically inducing analgesia.

### SUMMARY OF THE INVENTION

Accordingly, there are disclosed pharmaceutically acceptable compositions for otic and transdermal administration to an animal and methods for the use thereof. Such compositions comprise buprenorphine, a pharmaceutically acceptable carrier system comprising a solvent consisting of a water phase and organic phase, at least one penetration enhancing agent and, optionally, a stabilizing agent, a preservative, antioxidant, viscosity increasing agent and/or a tonicity adjustment agent. The present composition can also optionally include a non-opioid analgesic, such as a non-steroidal anti-inflammatory drug (NSAID), N-methyl-d-aspartate (NMDA) receptor antagonist, alpha-2 adrenergic receptor agonist, sodium channel blocker, or transient receptor potential (TRP) ion channel ligand.

With the foregoing and other objects, advantages and features of the invention that will become hereinafter apparent, the nature of the invention may be more clearly understood by reference to the following detailed description of the invention and the appended claims.

### BRIEF DESCRIPTION OF THE FIGURES

FIG. 1 is a graph showing the mean (±1 SD) plasma concentration of buprenorphine versus time in five healthy cats following otic administration of a buprenorphine formulation at a dose of 0.25-0.50 mg/kg.

FIG. 2 is a graph showing the mean pain assessed by Visual Analog Scale (VAS) for cats following declaw procedure and treatment with either subcutaneous meloxicam, an FDA-approved post-operative analgesic for cats, or otic buprenorphine. Meloxicam cats are represented by the broken line and have +1 SD shown for each time point. Buprenorphine cats are represented by the solid line and have −1 SD shown for each time point.

FIG. 3 is a graph showing the mean plasma concentration of buprenorphine versus time in six healthy cats following transdermal administration of a buprenorphine formulation at a dose of either 0.17-0.35 mg/kg or 0.35-0.70 mg/kg. Data for cats dosed at 0.17-0.35 mg/kg are represented by the broken line and have −1 SD shown for each timepoint; data for cats dosed at 0.35-0.70 mg/kg are represented by the solid line and have +1 SD shown for each time point.

### DETAILED DESCRIPTION OF THE INVENTION

It has been found that effective concentrations of opioids in the systemic circulation for the purpose of providing systemic analgesia can be achieved by the otic or transdermal routes of administration. By using the otic or transdermal route of administration, liver/gut wall (“first-pass”) metabolism of the opioid is avoided, which may enhance bioavailability relative to oral dosing.

The present invention relates to an opioid analgesic product for providing systemic analgesia, e.g., pre-emptive and perioperative analgesia, for mammals such as cats and dogs. The present invention comprises at least one opioid analgesic in a pharmaceutically acceptable vehicle. The compositions of the present invention can be used to simultaneously prevent or reduce the pain associated with surgery or injury. Use for the treatment of chronic pain associated with, e.g., neoplasia, osteoarthritis, pruritis, etc. is also contemplated.

The terms “otic” and “by ear” are used interchangeably herein to mean relating to the ear.

As used herein, “opiate” means any preparation or derivative of opium. The term “opioid” refers to both opiates and synthetic or semi-synthetic narcotics resembling opiates.

As used herein, the term “water phase” means a solvent system comprised of water, isotonic solution, a buffer system and/or any solvent mixable with water.

As used herein, the term “organic phase” means a solvent system comprised of any organic solvent or solvent system mixable or not mixable with water.

Active ingredients include opioid analgesics, in particular those having agonist activity at the mu opiate receptor, such as buprenorphine, morphine, diamorphine, meperidine, methadone, etorphine, levorphanol, fentanyl, alfentanil, sufentanil, oxycodone, hydrocodone, codeine, and oxymorphone. Particularly preferred is buprenorphine because of a wider safety margin and longer duration of activity.

In the preferred embodiment, the formulation is long acting, e.g. it is administered up to three times a day as needed. Because it is a long acting formulation, as opposed to a short acting formulation, one particular advantage of the present invention is the reduced dosing frequency, offering convenience for the person administering the product.

It will also be appreciated that the present invention encompasses, in one aspect, methods of alleviating pain by administering, for example, a pharmaceutically acceptable composition comprising, for example, buprenorphine, to an animal by otic or transdermal administration. Dosing administration may also be accomplished, for instance, by applying multiple or single drops to the ear or skin of the animal.

For example, plasma concentrations of buprenorphine, following single dose otic administration at a dose of about 0.05 to about 0.1 mg/kg there was achieved a Cmax of about greater than 5 ng/ml at a Tmax of about 60 minutes, and at a dose of about 0.1 to about 0.2 mg/kg, there was achieved a Cmax of about greater than 7 ng/ml at a Tmax of about 30 minutes.

In another example, plasma concentrations of buprenorphine, following single dose otic administration at a dose of about 0.3 to about 0.6 mg/kg there was achieved a Cmax of about 28 ng/ml at a Tmax of about 90 minutes, and at a dose of about 0.25 to about 0.5 mg/kg, there was achieved an initial peak of about greater than 10 ng/mL at about 30 minutes, followed by a Cmax of about greater than 12 ng/ml at a Tmax of about 2 hours.

In yet another embodiment, plasma concentrations of buprenorphine, following single dose transdermal administration at a dose of about 0.35 to about 0.70 mg/kg there was achieved a Cmax of about 10 ng/ml at a Tmax of about 30 minutes. When a dose of about 0.17 to about 0.35 mg/kg was used, there was achieved a Cmax of about greater than 3 ng/mL at a Tmax of about 4 hours.

Metabolites of opioid analgesics that have analgesic activity may also be used. Such metabolites include, e.g., analgesically active glucuronide and sulphate forms of opioids such as morphine-6-glucoronide.

Due to possible problems created by the unpleasant odor of the drug, low bioavailability, or the potential for local analgesic effect, it may be desirable to use a prodrug form of such opioid. Particularly preferred prodrug forms are those in which the 3-phenolic hydroxy group is esterified. Examples of prodrug derivatives suitable for use in the present invention include those disclosed in U.S. Pat. Nos. 4,668,685 and 4,673,679, both assigned to DuPont.

In another embodiment, the present invention allows for the inclusion of a non-opioid analgesic, such as an NSAID. Preferred NSAIDs, include acemetacin, acetylsalicylic acid (aspirin), alminoprofen, benoxaprofen, bucloxic acid, carprofen, celecoxib, clidanac, deracoxib, diclofenac, diflunisal, dipyrone, etodolac, fenoprofen, fentiazac, firocoxib, flobufen, flufenamic acid, flufenisal, flunixin, fluprofen, flurbiprofen, ibuprofen, indomethacin, indoprofen, isoxicam, ketoprofen, ketorolac, meclofenamic acid, mefenamic acid, meloxicam, miroprofen, nabumetone, naproxen, niflumic acid, oxaprozin, oxepinac, phenylbutazone, piroxicam, pirprofen, pramoprofen, sudoxicam, sulindac, suprofen, tepoxalin, tiaprofenic acid, tiopinac, tolfenamic acid, tolmetin, trioxaprofen, zidometacin, or zomepirac, pharmaceutically acceptable salts thereof and mixtures thereof. Particularly preferred NSAIDS include carprofen, deracoxib, etodolac, firocoxib, flunixin, ketoprofen, meloxicam and tepoxalin. Preferred NMDA receptor antagonists include memantine, ketamine, tiletamine, and pharmaceutically acceptable salts thereof and mixtures thereof. A particularly preferred NMDA receptor antagonist is ketamine. Preferred alpha-2 adrenergic receptor agonists include clonidine, detomidine, dexmedetomidine, fadolmidine, medetomidine, moxonidine, romifidine, xylazine, and pharmaceutically acceptable salts thereof and mixtures thereof. Particularly preferred alpha-2 adrenergic receptor agonists include detomidine and xylazine. Preferred sodium channel blockers include benzocaine, bupivacaine, lamotrigine, levobupivicaine, lidocaine, lignocaine, oxybuprocaine, prilocalne, proxymetacaine, ropivicaine, and pharmaceutically acceptable salts thereof and mixtures thereof. Particularly preferred sodium channel blockers include bupivacaine and lidocaine.

In general the formulations of the present invention will contain from about 0.1 to about 10% of the opioid(s) in an otically or transdermally acceptable vehicle. The amount of the opioid(s) may be varied to alter the dose volume and/or the dosage schedule. The amount of a second analgesic, such as an NSAID, will depend on synergy with the opioid and bioavailability and will be titrated to effect.

The compositions of the present invention may take various forms. For example, they may be a gel, liquid, or ointment.

The solvent used in the composition consists of a water and organic phase. Suitable solvents for the formulation include, but are not limited to, glyceryl formal, dimethylformamide, N-methyl-pyrrolidone, 2-pyrrolidone, glycol, propylene glycol, polyethylene glycol, diethylisosorbide, water, ethanol, isopropanol, 1,2-propanediol, glycerin, triethyl citrate, benzyl alcohol, dimethylisosorbide, C₂-C₉ alkylene diols, e.g., butylene diol, pentylene glycol, neopentyl diol, propylene glycol diethylene glycol, monoethyl ether or like compounds such as di C₂-C₅ alkylene diol, mono C₁-C₄ alkyl ethers, e.g., dipropylene glycol, mono propyl ether, mono propyl ether, and mono ethyl ether. Preferred solvents include 2-pyrrolidone, glyceryl formal, dimethylformamide, N-methyl-pyrrolidone, propylene glycol, polyethylene glycol, diethylisosorbide, ethanol, isopropanol, 1,2-propanediol, glycerin, triethyl citrate, benzyl alcohol, dimethylisosorbide and water. A particularly preferred solvent is propylene glycol.

Preferably, such a solvent is present in an amount of up to about 80% by weight of the formulation. More preferably, such a solvent is present at about 10% to about 75% of the formulation.

Suitable penetration enhancers may include lipophilic and/or hydrophilic components. Suitable penetration enhancers can be, for example, an alcohol, a nonionic solubilizer or an emulsifier. Suitable penetration enhancers include, but are not limited to, ethylene glycol, propylene glycol, dimethyl sulfoxide (DMSO), dimethyl polysiloxane (DMPX), oleic acid, caprylic acid, isopropyl alcohol, 1-octanol, ethanol (denatured or anhydrous), benzyl alcohol and other pharmaceutical grade or absolute alcohols with the exception of methanol. Other penetration enhancers include, water, sulphoxides and similar chemicals, such as DMSO, dimethylacetamide (DMA), dimethylformamide (DMF), etc., azone and related compounds, pyrrolidones, such as N-methyl-pyrrolidone (NMP), 2-pyrrolidone (2-pyrrol), etc., fatty alcohols, fatty acids and related structures, such as oleyl alcohol, oleic acid, linoleic acid, isopropyl myristate, etc., alcohols and glycols, such as ethanol, propylene glycol, lauryl alcohol esters, lauryl alcohol, etc., the esters of propylene glycol, such as propylene glycol monolaurate, surfactants, such as sodium lauryl sulphate (SLS), etc., urea, essential oils, terpenes and terpenoids, such as menthol, eucalyptus oil, 1,8-cineole, etc., phospholipids and solvents and related compounds, such as transcutol (ethoxydiglycol), etc. Preferred penetration enhancers are menthol, alcohols, benzyl alcohol, ethanol, water, glycols, esters of propylene glycol, propylene glycol monolaurate, lauryl alcohol esters or lauryl alcohol.

The viscosity of the vehicle may be increased or decreased as necessary by the use of various additional agents. The viscosity enhancing agent may be a water-dispersible acid polymer, a polysaccharide gum, and/or a mixture thereof. Suitable viscosity enhancing agents for use in the compositions of the present invention include, but are not limited to, polyvinyl alcohol, polyvinyl pyrrolidone magnesium sulfate, propylene glycol, lanolin, glycerin, hydroxypropylcellulose and other agents known to those skilled in the art to be suitable for use in the ear. A preferred viscosity enhancing agent is hydroxypropylcellulose.

Emulsifiers suitable for use in the compositions of the present invention include, e.g., polyethylene glycol (PEG) 30 dipolyhydroxystearate (e.g. ARLICEL P135, available from ICI Surfactants, Wilmington, Del.), PEG-40 stearate sorbitan oleate (e.g. CRILL 4, available from Croda, Inc., Parsippany, N.J.), polysorbate 80 (e.g. TWEEN 80, available from ICI Surfactants.

One component of the organic solution is a solvent composed of compounds, such as suitable surfactants for the organic solution, which include, but are not limited to, monoglycerides or like compounds such as glyceryl mono-oleate, -laurate, -behenate, -eicosadioate, -sterate, or other fatty acid mono substituted glycerides.

Suitable film formers for the organic solution include, but are not limited to, polyacrylamide or other like compounds, which act as thickening agents such as other acrylamide copolymers, polyacrylate copolymers, cellulosic polymers and copolymers, and polyvinyl pyrrolidone polymers and copolymers.

Other optional inert ingredients may be added to the present composition, as desired. Such ingredients include, but are not limited to, preservatives, chelating agents, antioxidants and stabilizers. Exemplary preservatives include, but are not limited to, BHT, methyl p-hydroxybenzoate (methylparaben) and propyl p-hydroxybenzoate (propylparaben). It will also be appreciated that the formulations of the present invention in another embodiment are self-preserving. Exemplary chelating agents include, but are not limited to, edetate sodium. Exemplary antioxidants include, but are not limited to, butylated hydroxyanisole (BHA), BHT and sodium monothioglycerol. Preferred stabilizers to prevent degradation of any of the active ingredients in the formulations of the present invention include, but are not limited to, BHT, BHA, sodium monothiogylcerol, methyl p-hydroxybenzoate (methylparaben) and propyl p-hydroxybenzoate (propylparaben). Other preferred stabilizers include, but are not limited to, triethyl citrate, USP, NF, acetic acid, glacial acetic acid, fumaric acid, hydrochloric acid, diluted hydrochloric acid, malic acid, nitric acid phosphoric acid, diluted phosphoric acid, sulfuric acid and tartaric acid. Particularly preferred stabilizers for use in the present invention include, but are not limited to, BHT, BHA, sodium monothioglycerol or citric acid in a concentration of about 5% or less and monothioglycerol in a concentration of about 0.1% to 2% w/v.

Preferably the pH of the compositions of the present invention is adjusted to maintain buprenorphine or buprenorphine HCl in solution. Preferably, the pH of the compositions of the present invention are between about 3 and about 10, preferably about 3.5 to about 6. An appropriate buffering agent may be added to maintain the pH. Suitable buffers include, but are not limited to, potassium chloride, sodium or potassium phosphates (monobasic and dibasic), sodium or potassium acetates, sodium or potassium borates (e.g., sodium tetraborate decahydrate), sodium or potassium citrates, sodium or potassium hydroxides and equivalents or mixtures thereof, and weak acids, such as acetic, boric, and phosphoric acids.

In order to prepare the composition of the present invention, the vehicle(s) or a portion of the vehicle(s), are added to the compounding vessel, followed by the remaining excipients and the actives. The mixture is mixed until all solids are dissolved or in suspension. Additional solvent(s) to bring the composition to final volume may be added if needed. Additives, such as those listed above, may also be included in the vessel and mixed into the formulation (the order of addition is not critical).

After application of the formulation, the opioid present in the composition is systemically absorbed. It is an advantage of the method of the present invention that it can provide a rapid initial absorption with some delayed release for continuous absorption of the active drug, thereby providing a better pharmacokinetic profile than intravenous or other parenteral routes for dosing. Onset of analgesic action after administration of the compositions of the present invention begins within 30 minutes of application, and the duration of analgesic action generally lasts up to at least 8 hours.

Another advantage of the present invention is that some of the formulations appear to have a rapid absorptive phase and a prolonged plateau phase (slow absorptive phase). Thus, the above desirable characteristics can be achieved with one formulation. Other advantages of the present invention are the fact that animals in pain and/or animals on an opiate can be aggressive. Therefore, administration of the present invention has the advantage that an animal handler never has to go near the mouth/teeth of the animal, i.e., increased animal handler safety.

The method of the present invention, and the formulations to carry out the method, have other advantages over existing products, such as ease of administration for both the veterinary staff and the owner of the animal, reduction in side effects, etc. In the case of an adverse event, the activity of the opioid is reversible by administration of opioid antagonists, e.g. naloxone.

It is believed that the route of administration may improve the bioavailability of many analgesic agents such as opioids that undergo hepatic first-pass metabolism and gastrointestinal degradation when administered orally. It is possible that the metabolism of such compounds may be favorably affected by the route of administration.

The appropriate dosage can be determined according to the weight of the animal. As will be appreciated by one of skill in the art, if renal or hepatic function is compromised, drug dosage may need to be decreased to account for decreased elimination.

The compositions of the present invention may be packaged in many forms. Preferably the formulation is packaged as single-dose, single-use units. Such single-dose packaging overcomes problems of bacterial contamination of multiple-dose preparations and minimizes the likelihood of accidental acute overdosing.

The following examples are given for the purpose of illustrating the present invention and should not be construed as limiting the scope or spirit of the invention.

### EXAMPLE 1

### Otic

| Ingredient | Conc w/w |
|---|---|
| Buprenorphine HCl | 1.62% |
| (Free Base Equivalent) | (1.50%) |
| Hydroxypropylcellulose GF | 0.50% |
| Benzyl Alcohol | 5% |
| Purified water | 20% |
| BHT | 0.05% |
| Alcohol USP/BP 200 proof | 15% |
| Propylene glycol monolaurate | 20% |
| Propylene glycol, qs | 38% |

This Example may be prepared according to customary procedures known to one of skill in the art. In a specific embodiment, the formulation can be prepared and stored in two different solvent systems consisting of an organic phase system and a water phase system to be combined to obtain the final formulation.

### EXAMPLE 2

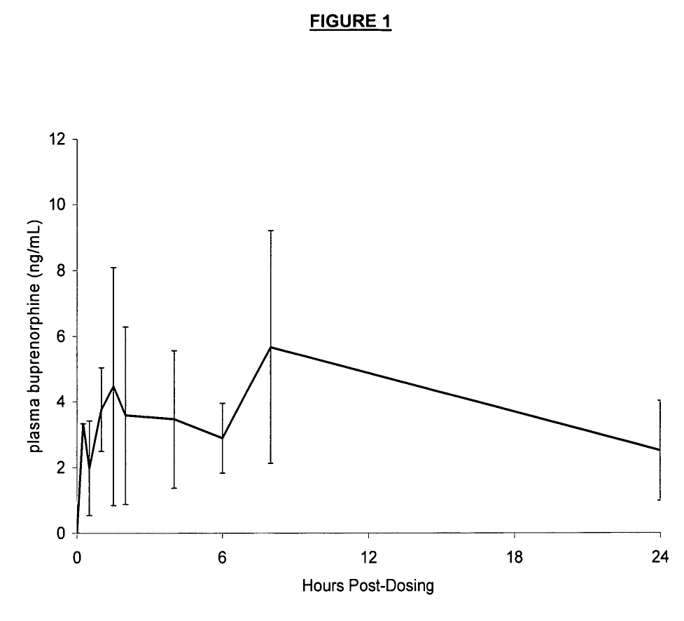

Five healthy cats were administered the formulation in Example 1 at a dosage of 0.25-0.50 mg/kg. Serial blood samples were drawn at time 0 prior to dosing, then at 0.25, 0.5, 1, 1.5, 2, 4, 6, 8, and 24 hours after dosing. Plasma concentrations (ng/mL) of buprenorphine versus time were reported and graphically presented. The results are shown in FIG. 1. Two plasma peaks are evident—the first of about 4 ng/mL occurs at 90 minutes, while the second of about 5 ng/mL occurs at 8 hours.

These data display that the formulation described in Example 1 has a benefit, in that buprenorphine is detectable in plasma shortly after dosing, suggesting that analgesia will occur early. Secondly, the plasma peak occurs at about 8 hours after dosing, suggesting that analgesia will be long-lasting.

### EXAMPLE 3

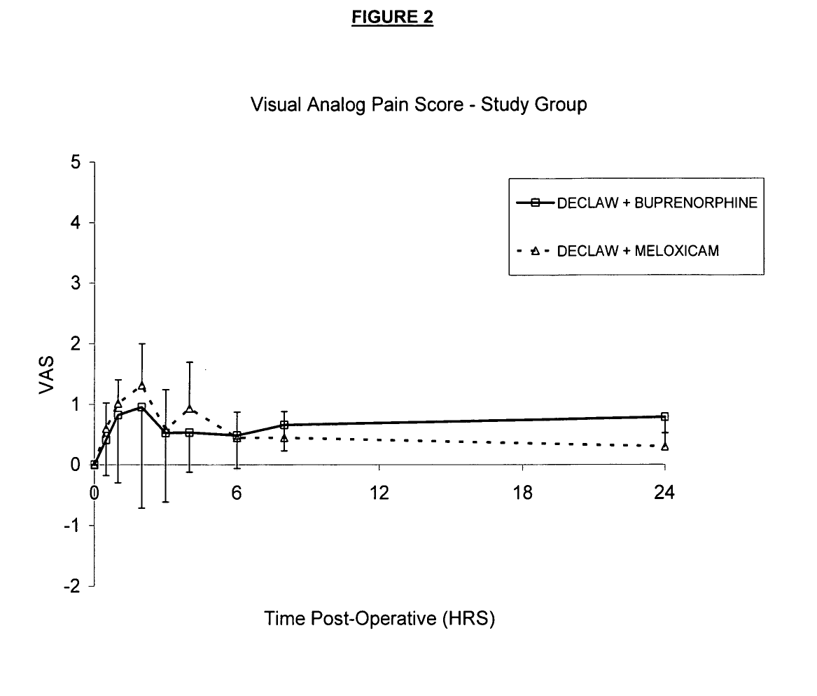

Fourteen healthy cats were used in a study described below to evaluate the analgesic properties of the formulation described in Example 1. The cats were placed under general anesthesia and had bilateral forelimb onychectomy (declaw) performed by a licensed veterinarian. Prior to induction of anesthesia, six of the cats received a subcutaneous injection (0.3 mg/kg) of meloxicam, which is approved in the United States for post-operative analgesia in cats. Eight cats were dosed with 0.6 mg/kg of the buprenorphine formulation described in Example 1. Following surgery, all cats were evaluated for signs of pain using a Visual Analog Scale (VAS) at 0.5, 1, 2, 3, 4, 6, 8, and 24 hours. The mean VAS versus time post-surgery for cats treated with meloxicam or with buprenorphine was reported and graphically represented. The results are shown in FIG. 2.

These data suggest that the post-surgical analgesic profile of the formulation described in Example 1 is similar to that of an FDA-approved post-operative analgesic for cats.

### EXAMPLE 4

### Otic

| Ingredient | Conc w/w |
|---|---|
| Buprenorphine HCl | 1.62% |
| (Free Base Equivalent) | (1.50%) |
| Hydroxypropylcellulose GF | 0.50% |
| Benzyl Alcohol | 5% |
| BHT | 0.05% |
| Alcohol USP/BP 200 proof | 15% |
| Lauryl Alcohol | 20% |
| Propylene glycol, qs | 58% |

This Example may be prepared according to customary procedures known to one of skill in the art. In a specific embodiment, the formulation can be prepared and stored in two different solvent systems consisting of an organic phase system and a water phase system to be combined to obtain the final formulation.

### EXAMPLE 5

### Topical/Transdermal

| Ingredient | Conc w/w |
|---|---|
| Buprenorphine HCl | 2.16% |
| (Free Base Equivalent) | (2.00%) |
| Hydroxypropylcellulose GF | 0.30% |
| Benzyl Alcohol | 10% |
| BHT | 0.05% |
| Alcohol USP/BP 200 proof | 20% |
| Levo Menthol USP/EP | 8% |
| Purified Water | 10% |
| Propylene glycol, qs | qs |

This Example may be prepared according to customary procedures known to one of skill in the art. In a specific embodiment the formulation can be prepared and stored in two different solvent systems consisting of an organic phase system and a water phase system to be combined to obtain the final formulation

### EXAMPLE 6

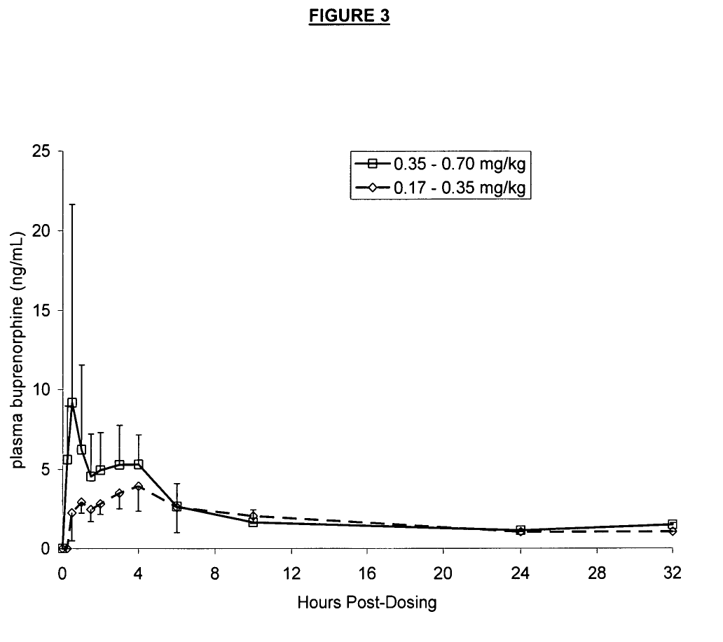

Six healthy cats were administered the formulation in Example 5 once using a dosage of 0.17-0.35 mg/kg, and then again using a dosage of 0.35-0.70 mg/kg. Following each dosing, serial blood samples were drawn at time 0 prior to dosing, then at 0.25, 0.5, 1, 1.5, 2, 3, 4, 6, 10, 24, and 32 hours after dosing. Plasma concentrations (ng/mL) of buprenorphine versus time were reported and graphically presented. The results are shown in FIG. 3.

These data display that the formulation described in Example 5 has a benefit, in that buprenorphine is detectable in plasma shortly after transdermal dosing, suggesting that analgesia will occur early. Secondly, plasma levels are detectable for as long as 32 hours following dosing, suggesting that analgesia will be long-lasting.

Although certain presently preferred embodiments of the invention have been described herein, it will be apparent to those skilled in the art to which the invention pertains that variations and modifications of the described embodiment may be made without departing from the spirit and scope of the invention. Accordingly, it is intended that the invention be limited only to the extent required by the appended claims and the applicable rules of law.

We claim:

1. A pharmaceutically acceptable composition for otic administration to an animal comprising buprenorphine, a pharmaceutically acceptable carrier system comprising a solvent consisting of a water phase and organic phase, and at least one penetration enhancing agent.

2. The pharmaceutically acceptable composition according to claim 1, wherein the penetration enhancing agent includes lipophilic and/or hydrophilic components.

3. The pharmaceutically acceptable composition according to claim 2, wherein the penetration enhancing agent is selected from the group consisting of esters of propylene glycol, menthol, alcohol, glycols, or water in an amount sufficient to enhance penetration of the buprenorphine.

4. The pharmaceutically acceptable composition according to claim 3, wherein the glycol is propylene glycol monolaurate.

5. The pharmaceutically acceptable composition according to claim 3, wherein the alcohol is selected from the group consisting of ethanol, lauryl alcohol and lauryl alcohol esters.

6. The pharmaceutically acceptable composition according to claim 5, wherein the alcohol is an ester of lauryl alcohol.

7. The pharmaceutically acceptable composition according to claim 1, wherein the solvent is selected from the group consisting of 2-pyrrolidone, glyceryl formal, dimethylformamide, N-methyl-pyrrolidone, propylene glycol, polyethylene glycol, diethylisosorbide, ethanol, isopropanol, 1,2-propanediol, glycerin, triethyl citrate, benzyl alcohol, dimethylisosorbide and water.

8. The pharmaceutically acceptable composition according to claim 7, wherein the solvent is propylene glycol.

9. The pharmaceutically acceptable composition according to claim 1, wherein the composition has a pH range of about 3 to about 10.

10. The pharmaceutically acceptable composition according to claim 1, further comprising a stabilizing agent.

11. The pharmaceutically acceptable composition according to claim 10, wherein the stabilizing agent is selected from the group consisting of BHT, BHA and sodium monothioglycerol.

12. The pharmaceutically acceptable composition according to claim 1, further comprising a preservative agent.

13. The pharmaceutically acceptable composition according to claim 12, wherein the preservative agent is selected from the group consisting of BHT, methyl p-hydroxybenzoate (methylparaben) and propyl p-hydroxybenzoate (propylparaben).

14. The pharmaceutically acceptable composition according to claim 1, further comprising a viscosity enhancing agent and/or and antioxidant agent.

15. The pharmaceutically acceptable composition according to claim 14, wherein the viscosity enhancing agent is selected from the group consisting of a water-dispersible acid polymer, a polysaccharide gum, and/or a mixture thereof.

16. The pharmaceutically acceptable composition according to claim 15, wherein the viscosity enhancing agent is hydroxypropylcellulose.

17. The pharmaceutically acceptable composition according to claim 14, wherein the antioxidant agent is selected from the group consisting of BHT, BHA and sodium monothioglycerol.

18. The pharmaceutically acceptable composition according to claim 1, further comprising a non-opioid analgesic.

19. The pharmaceutically acceptable composition according to claim 18, wherein the non-opioid analgesic is selected from the group consisting of acemetacin, acetylsalicylic acid (aspirin), alminoprofen, benoxaprofen, bucloxic acid, carprofen, celecoxib, clidanac, deracoxib, diclofenac, diflunisal, dipyrone, etodolac, fenoprofen, fentiazac, firocoxib, flobufen, flufenamic acid, flufenisal, flunixin, fluprofen, flurbiprofen, indoprofen, isoxicam, ketoprofen, ketorolac, meclofenamic acid, mefenamic acid, meloxicam, miroprofen, nabumetone, naproxen, niflumic acid, oxaprozin, oxepinac, phenylbutazone, piroxicam, pirprofen, pramoprofen, sudoxicam, sulindac, suprofen, tepoxalin, tiaprofenic acid, tiopinac, tolfenamic acid, tolmetin, trioxaprofen, zidometacin, zomepirac, and pharmaceutically acceptable salts thereof and mixtures thereof.

20. A method for inducing analgesia in an animal by otically administering buprenorphine in the pharmaceutically acceptable composition of claim 1.

21. A method for inducing a systemic analgesic effect in an animal by otically administering buprenorphine.

22. The method of claim 21, wherein the analgesic effect is for at least about 8 hours.

23. A method for inducing systemic analgesic effect in an animal by otically administering buprenorphine, wherein at a dosing range of about 0.05 to about 0.6 mg/kg there is achieved a buprenorphine level in the animal of a Cmax of in a range of about 5 ng/ml to about 28 ng/ml and a Tmax in the range of about 0.5 hours to about 2 hours.

24. A pharmaceutically acceptable composition for transdermal administration to an animal comprising buprenorphine, a pharmaceutically acceptable carrier system comprising a solvent consisting of a water phase and organic phase, and at least one penetration enhancing agent.

25. The pharmaceutically acceptable composition according to claim 24, wherein the penetration enhancing agent includes lipophilic and/or hydrophilic components.

26. The pharmaceutically acceptable composition according to claim 25, wherein the penetration enhancing agent is selected from the group consisting of esters of propylene glycol, menthol, alcohol, glycols and water in an amount sufficient to enhance penetration of the buprenorphine.

27. The pharmaceutically acceptable composition according to claim 26, wherein the glycol is propylene glycol monolaurate.

28. The pharmaceutically acceptable composition according to claim 26, wherein the alcohol is selected from the group consisting of ethanol, lauryl alcohol and lauryl alcohol esters.

29. The pharmaceutically acceptable composition according to claim 26, wherein the penetration enhancing agent is menthol.

30. The pharmaceutically acceptable composition according to claim 24, wherein the solvent is selected from the group consisting of 2-pyrrolidone, glyceryl formal, dimethylformamide, N-methyl-pyrrolidone, propylene glycol, polyethylene glycol, diethylisosorbide, ethanol, isopropanol, 1,2-propanediol, glycerin, triethyl citrate, benzyl alcohol, dimethylisosorbide and water.

31. The pharmaceutically acceptable composition according to claim 30, wherein the solvent is propylene glycol.

32. The pharmaceutically acceptable composition according to claim 24, wherein the composition has a pH in the range of about 3 to about 10.

33. The pharmaceutically acceptable composition according to claim 24, further comprising a viscosity enhancing agent, a preservative and/or antioxidant agent.

34. The pharmaceutically acceptable composition according to claim 33, wherein the viscosity enhancing agent is selected from the group consisting of a water-dispersible acid polymer, a polysaccharide gum, and/or a mixture thereof.

35. The pharmaceutically acceptable composition according to claim 34, wherein the viscosity enhancing agent is hydroxypropylcellulose.

36. The pharmaceutically acceptable composition according to claim 33, wherein the preservative agent is selected from the group consisting of BHT, methyl p-hydroxybenzoate (methylparaben) and propyl p-hydroxybenzoate (propylparaben).

37. The pharmaceutically acceptable composition according to claim 33, wherein the antioxidant agent is selected from the group consisting of BHT, BHA and sodium monothioglycerol.

38. The pharmaceutically acceptable composition according to claim 24, further comprising a non-opioid analgesic.

39. The pharmaceutically acceptable composition according to claim 38, wherein the non-opioid analgesic is selected from the group consisting of acemetacin, acetylsalicylic acid (aspirin), alminoprofen, benoxaprofen, bucloxic acid, carprofen, celecoxib, clidanac, deracoxib, diclofenac, diflunisal, dipyrone, etodolac, fenoprofen, fentiazac, firocoxib, flobufen, flufenamic acid, flufenisal, flunixin, fluprofen, flurbiprofen, indoprofen, isoxicam, ketoprofen, ketorolac, meclofenamic acid, mefenamic acid, meloxicam, miroprofen, nabumetone, naproxen, niflumic acid, oxaprozin, oxepinac, phenylbutazone, piroxicam, pirprofen, pramoprofen, sudoxicam, sulindac, suprofen, tepoxalin, tiaprofenic acid, tiopinac, tolfenamic acid, tolmetin, trioxaprofen, zidometacin, zomepirac and pharmaceutically acceptable salts thereof and mixtures thereof.

40. A method for inducing analgesia in an animal by transdermally administering buprenorphine in the pharmaceutically acceptable composition of claim 24.

41. A method for inducing a systemic analgesic effect in an animal by transdermally administering buprenorphine.

42. The method of claim 41, wherein the analgesic effect is for at least about 8 hours.

43. A method for inducing analgesia in an animal by transdermally administering buprenorphine, wherein at a dosing range of about 0.17 to about 0.70 mg/kg there is achieved a buprenorphine level in the animal of a Cmax of about 3 to about 10 ng/mL and a Tmax of about 30 minutes to about 4 hours.
